(19) United States
(12) Patent Application Publication
(10) Pub. No.: US 2026/0294493 A1
(43) Pub. Date: Oct. 1, 2026

(54) CONNECTING AND FIXING STRUCTURE OF FUNNEL CHEST CORRECTION PLATE

(71) Applicant: PTS Technology CO., LTD., New Taipei City (TW)

(72) Inventor: YI-TAI CHIEN, New Taipei City (TW)

(21) Appl. No.: 19/091,832

(22) Filed: Mar. 27, 2025

(51) Int. Cl. A61B 17/80; A61B 17/68

(52) U.S. Cl. CPC A61B 17/8076; A61B 2017/681

(57) ABSTRACT

A connecting and fixing structure of a funnel chest correction plate includes a fixing plate, a stop limiting portion, a fixing groove, free ends, an opening end, a clamp limiting portion, an open end, a fixing portion, a correction plate, an adjusting hole, and a fastening member. To combine the correction plate and the fixing plate, the correction plate is directly placed, through the open end of the fixing plate, on the fixing plate and entering through the opening end into the fixing groove, so that the stop limiting portion and the clamp limiting portion temporarily hold the correction plate and the fixing plate lying flat on each other. Then, the position of the correction plate is adjusted through the free ends to have the adjusting hole and the fixing portion aligned with each other. The fastening member sets the correction plate on the fixing plate in a movable manner.

### BACKGROUND OF THE INVENTION

### (a) Technical Field of the Invention

The present invention provides a connecting and fixing structure of funnel chest correction plate, which simplifies the operation of combining a correction plate and a fixing plate, and at the same time, takes into account convenience of disassembly and assembly of the correction plate, degree of freedom of movement, difficulty of production, structural strength, and overall health and safety.

### (b) Description of the Prior Art

The popularity of minimally invasive surgery has made it possible to treat a chest disease called funnel chest by custom-making and re-implanting metal plates of different sizes as the body grows.

As shown in FIG. 1, which is an exploded view of a known funnel chest correction plate, Taiwan Patent No. I627934 discloses a “Funnel Chest Self-Adjusting Correction System”, which allows automatic adjustment of the position of a metal plate in accordance with breathing. There are still problems such as “the first and second fixing units 91, 92 being relatively large in size, resulting in enlargement of the wound size in minimally invasive surgery to correspond thereto”, “the closed design of the first and second grooves 911, 921 creating a dead cavity that cannot be cleaned, so that it is easy for bacteria to grow and even get infected due to the accumulation of blood and other filth”, and “the first and second fixing units 91, 92 must be assembled from the ends of the correction plate 93, which not only requires a larger surgical incision, but also has inconvenience in operation”.

As shown in FIG. 2, which is an exploded view of a known funnel chest correction plate, Taiwan Patent No. I699185 discloses a “Structure of funnel chest correction plate”, which solves the above-mentioned problem, but the arrangement of the guide grooves 931 on both sides of the correction plate 93 leads to a significant increase in the manufacturing cost and production difficulty of the correction plate 93. In addition, the existence of the guide grooves 931 is set at such a location as to reduce the structural strength of the correction plate 93.

### SUMMARY OF THE INVENTION

The primary objective of the present invention is that through utilizing a stop limiting portion and a clamp limiting portion provided on a fixing plate and allowing the correction plate to take into account difficulty of manufacturing and structural strength, the operation of combining the two can be simplified and convenience of disassembly and assembly is enhanced.

Another objective of the present invention is that through utilizing a movable and open way of combination, the degree of freedom of movement of the correction plate and hygiene and safety can both be taken into account.

To achieve the above objectives, a structure of the present invention comprises: at least one fixing plate, a stop limiting portion, a fixing groove, a plurality of free ends, an opening end, a clamp limiting portion, an open end, a fixing portion, a correction plate, an adjusting hole, and a fastening member, wherein the stop limiting portion is formed in the fixing plate; the fixing groove is defined between the fixing plate and the stop limiting portion; the free ends are respectively defined in two end surfaces of the fixing groove; the opening end is formed on one side of the fixing groove different from the free ends; the clamp limiting portion is formed on the fixing plate and is located at one side opposite to the stop limiting portion; the open end is defined between the clamp limiting portion and the stop limiting portion; the fixing portion is formed on the fixing plate and is located between the stop limiting portion and the clamp limiting portion; the correction plate is movable through the open end into the fixing groove to be disposed on the fixing plate; the adjusting hole is formed in the correction plate and corresponds in position to the fixing portion; and the fastening member corresponds to and is combinable with the adjusting hole and the fixing portion.

For a user using the present invention to treat funnel chest, it only needs to insert the correction plate and then insert the fixing plate through a small wound, so that to have the two combined together, the correction plate can be placed on the fixing plate through the open end of the fixing plate and then entering the fixing groove through the opening end to have the correction plate and the fixing plate temporarily lying flat against each other by means of the stop limiting portion and the clamp limiting portion to be then adjusting the position of the correction plate through the free ends to make the adjusting hole and the fixing portion aligned with each other, and the fastening member is applied to set the correction plate on the fixing plate in a movable manner, thereby completing the combination of the correction body and the fixing plate simply and quickly, and providing a certain space of movement for the correction plate to allow the correction plate to move with breathing, and reducing the size of the surgical wound. Also, the open type fixing groove can help avoid infection problems.

The above technology can overcome the problems of the conventional funnel chest correction plate, such as large wound area, infection risk, inconvenient operation, and high production cost of correction plates with guide groove, and achieve the above advantages of practical progress.

### BRIEF DESCRIPTION OF THE DRAWINGS

FIG. 1 is an exploded view showing a known funnel chest correction plate.

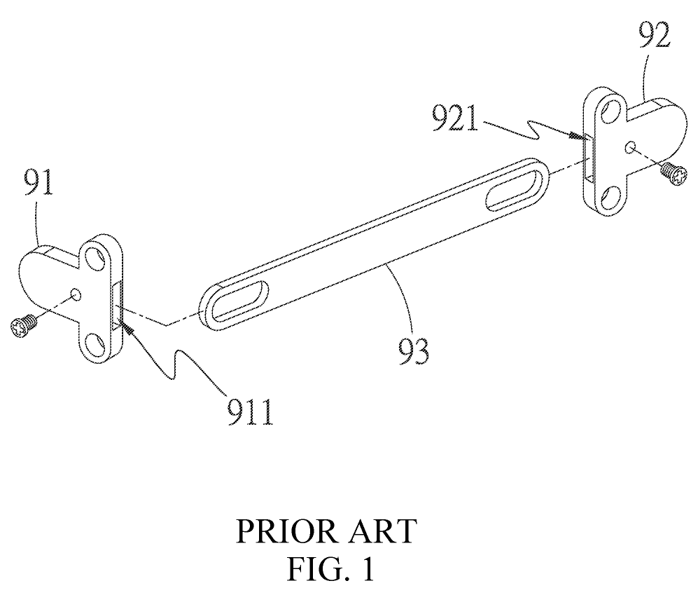

FIG. 2 is an exploded view showing a known funnel chest correction plate.

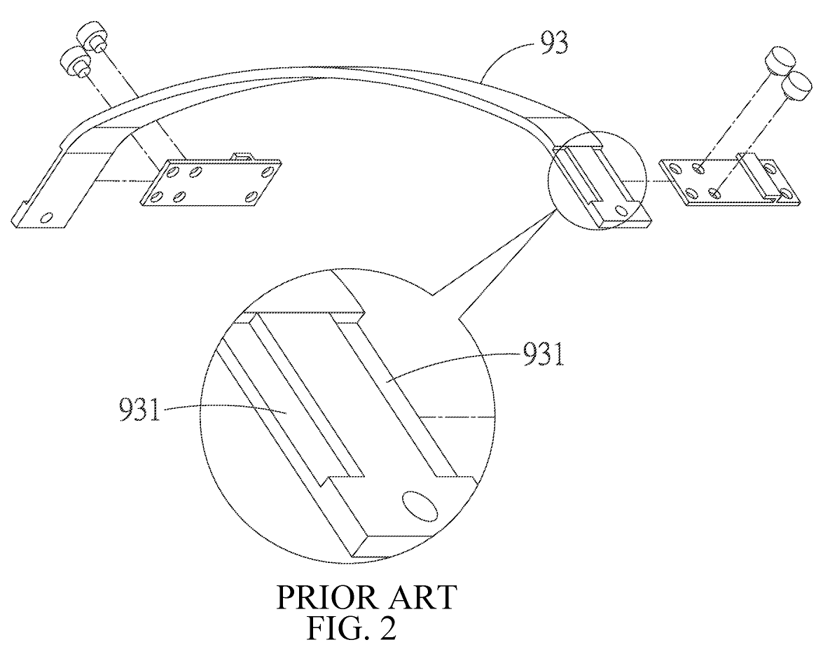

FIG. 3 is a perspective view showing a preferred embodiment according to the present invention.

FIG. 4 is an exploded view showing the preferred embodiment according to the present invention.

FIG. 5 is a plan view showing a fixing plate of the preferred embodiment according to the present invention.

FIG. 6 is a schematic view illustrating implementation of the preferred embodiment according to the present invention.

FIG. 7 is a schematic view illustrating combining of the preferred embodiment according to the present invention.

FIG. 8 is a schematic view illustrating positioning of the preferred embodiment according to the present invention.

FIG. 9 is a schematic view illustrating fixing of another preferred embodiment according to the present invention.

FIG. 10 is a schematic view illustrating fixing of another preferred embodiment according to the present invention.

### DETAILED DESCRIPTION OF THE PREFERRED EMBODIMENTS

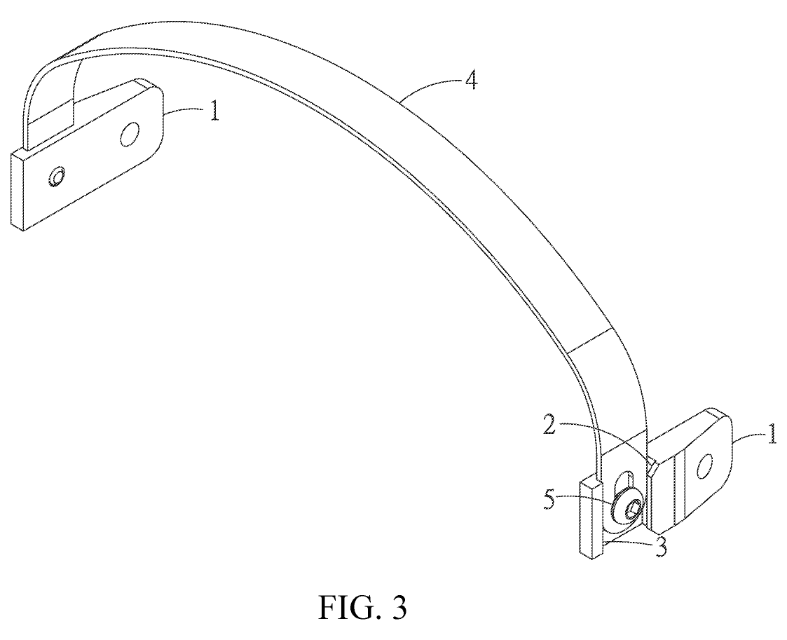

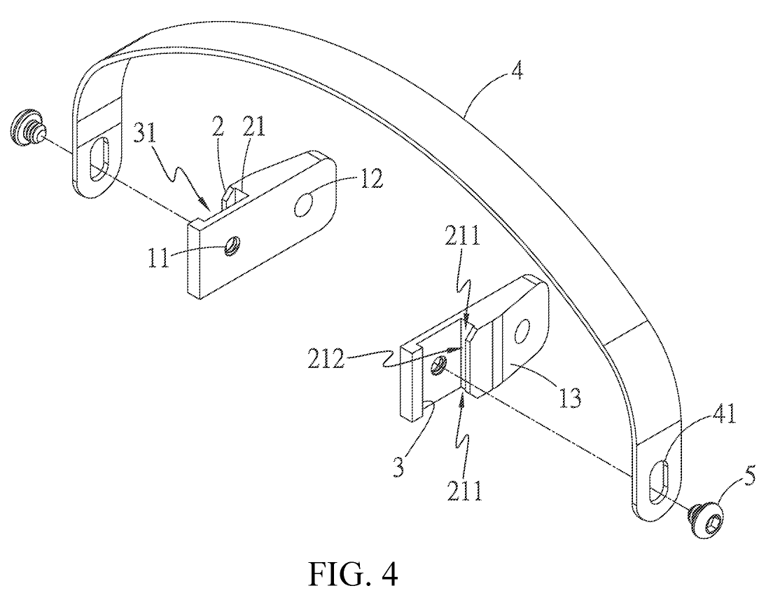

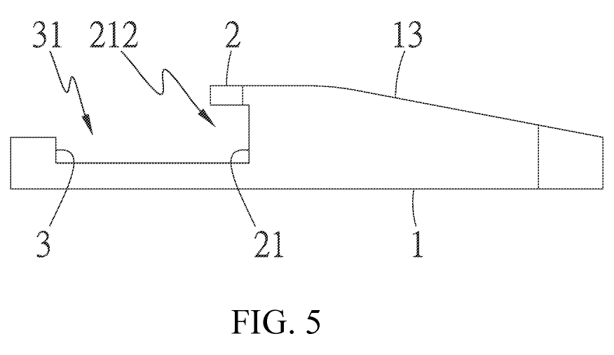

Referring to FIGS. 3 to 5, which are a perspective view of a preferred embodiment according to the present invention to a plan view of a fixing plate, it can be clearly seen from the drawings that the present invention comprises:

at least one fixing plate 1;

a stop limiting portion 2, which is formed on the fixing plate 1;

a fixing groove 21, which is defined between the fixing plate 1 and the stop limiting portion 2;

a plurality of free ends 211, which are respectively defined in two end surfaces of the fixing groove 21;

an opening end 212, which is formed on one side of the fixing groove 21 different from the free ends 211;

a clamp limiting portion 3, which is formed on the fixing plate 1 and is located at one side opposite to the stop limiting portion 2;

an open end 31, which is defined between the clamp limiting portion 3 and the stop limiting portion 2;

a fixing portion 11, which is formed on the fixing plate 1 and is located between the stop limiting portion 2 and the clamp limiting portion 3;

a correction plate 4, which is movable through the open end 31 into the fixing groove 21 to be disposed on the fixing plate 1 and clamped and limited by the stop limiting portion 2 and the clamp limiting portion 3;

an adjusting hole 41, which is formed in the correction plate 4 and corresponds in position to the fixing portion 11 when the correction plate 4 is disposed on the fixing plate 1; and

a fastening member 5, which corresponds to and is combinable with the adjusting hole 41 and the fixing portion 11.

The fixing plate 1 is formed with a through-hole portion 12 for positioning relative to ribs.

The fixing plate 1 has a smooth surface 13 located at one side of the stop limiting portion 2 opposite to the fixing groove 21.

The adjusting hole 41 is of an elongated shape to allow the fastening member 5 to move in a length direction of the correction plate 4.

The fixing plate 1 and the correction plate 4 are metal plates made of stainless steel, pure titanium, titanium alloys, or cobalt-chromium alloys, which will not rust even if placed in the human body for a long time. The stop limiting portion 2 is an inverted L-shaped structure formed on the fixing plate 1 or an eaves-like structure combined with the smooth surface 13 to form an upper edge thereof, and in the instant embodiment, the latter is taken as an example for illustration, The fixing portion 11 can be a screw hole having an internal thread, or a tight hole tight connection with the end of the fastening member 5, or a raised peg inserted into the adjusting hole 41, and in the case that the fixing portion 11 is a screw hole, the fastening member 5 is a screw, and in the case that the fixing portion 11 is a tight hole, the fastening member 5 is a tenon, and in the case that the fixing portion 11 is a raised peg, the fastening member is a nut or a collar, and in the instant embodiment, the fixing portion 11 being a screw hole is taken as an example for illustration. The adjusting hole 41 and the through-hole portion 12 are both holes having smooth inside surfaces. However, the corresponding types of the above-mentioned components are only examples of preferred embodiments. All types with the same functions belong to the scope of the present invention and are not limited to the above-mentioned examples.

Through the above description, the structure of this technology can be understood, and according to the corresponding cooperation of this structure, the operation of combining the correction plate 4 and the fixing plate 1 can be simplified, and at the same time, various advantages with regard to convenience of disassembly and assembly of the correction plate 4, degree of freedom of movement, difficulty of production, structural strength, and overall health and safety are all taken into account. A detailed explanation will be described below.

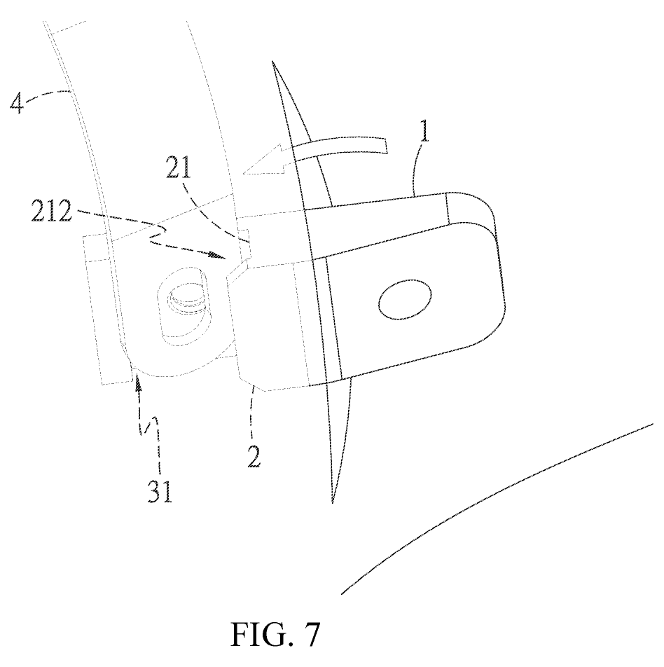

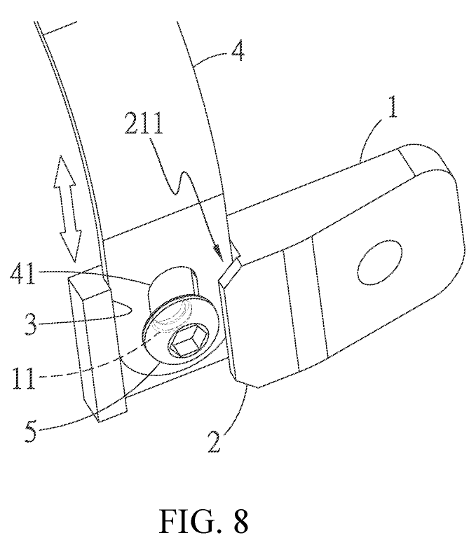

Referring to FIGS. 3-8, which are a perspective view of the preferred embodiment according to the present invention to a schematic view illustrating positioning of the preferred embodiment according to the present invention, when the above components are assembled, it can be clearly seen from the drawings that the fixing plate 1 of the present invention defines a free end 211 at each of two end surfaces of the fixing groove 21, and also has an opening end 212 on the side away from the bottom of the groove, so that the fixing groove 21 is set in an open state. In addition to allowing the operation of combining the correction plate body 4 not to be performed from the end to exhibit a high degree of convenience for disassembly and assembly, it can also greatly reduce the risk of accumulation of filth such as blood and water, and provide higher hygiene safety.

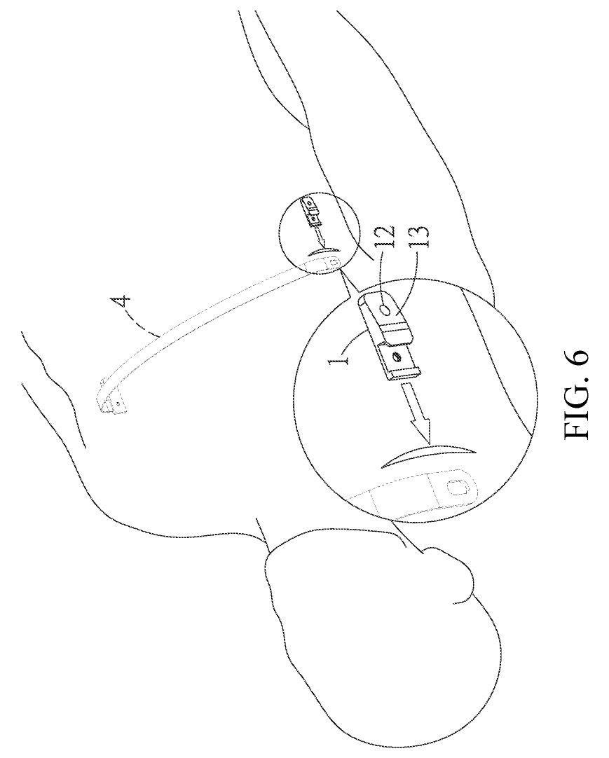

To insert the fixing plate 1, it only needs to make a small wound on the skin surface (as shown in FIG. 6), and the fixing plate 1 possess a design including a smooth surface 13 on one side to avoid scratching the skin or internal tissues. The through-hole portion 12 on the fixing plate 1 can be used to fix on the ribs after insertion. When the fixing plate 1 and the correction plate 4 are combined, the correction plate 4 can simply place the side wall of a long side thereof on the fixing plate 1 from the open end 31 of the fixing plate 1, and slide into the fixing groove 21 through the opening end 212. When sliding into the fixing groove 21, the correction plate 4 is subject to a limiting effect of the stop limiting portion 2 from top to bottom, so as to naturally lie flat on the fixing plate 1. At this time, the correction plate 4 and the fixing plate 1 are temporarily held in a state of being laid flat against each other by the stop limiting portion 2 and the clamp limiting portion 3, and then the position of the correction plate 4 is adjusted through the free ends 211 so as to have the adjusting hole 41 aligned with the fixing portion 11, and then, the correction plate 4 is movably set on the fixing plate 1 by means of the fastening member 5 to thus complete the operation of fixing. The overall operation is simple and convenient, and because the correction plate 4 has no other missing corner design except the opening of the adjusting hole 41, the structure is complete, so that the structural strength is greatly improved, and it is not easy to accumulate dirt. Moreover, because the adjusting hole 41 is of an elongated form, the fastening member 5 can move along the length direction of the correction plate 4, so that a movement space is provided for movement of the correction plate 4 when the user is breathing or growing, and also, the open type arrangement of the fixing groove 21 can greatly reduce the infection problem.

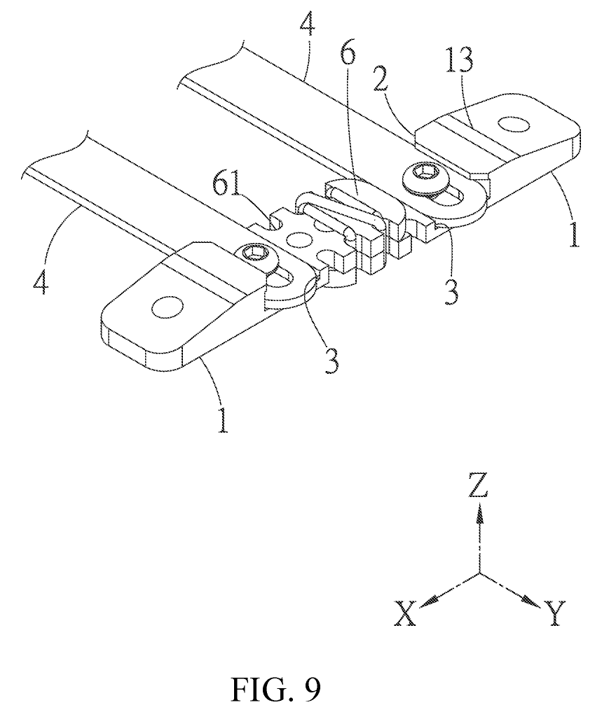

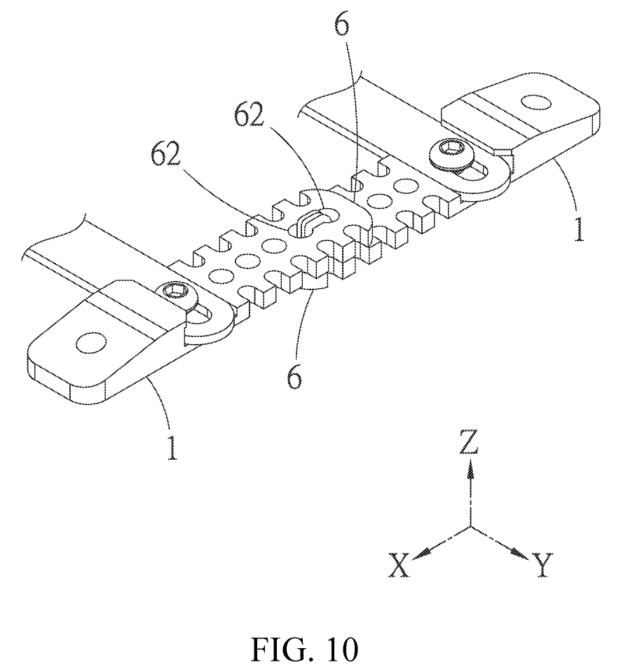

Referring further to FIGS. 9 and 10, which are two schematic views illustrating fixing according to another preferred embodiment of the present invention, it can be clearly seen from the drawings that the instant embodiment is similar to the previous embodiment and only further includes an extension board 6 extending from one side of the clamp limiting portion 3 and having a thickness less than the thickness from the fixing plate 1 to the stop limiting portion 2, and the extension board 6 is provided, on at least one side thereof, with least one engagement notch portion 61, or further including at least one connection aperture 62. A preferred thickness of the extension board 6 is one half of the thickness the fixing plate 1 (the thickness including the smooth surface 13), in order to reduce the overall thickness when two fixing plates 1 are used in a stacked form, or to reduce the extent of protruding when stacked in an inclined form, that is when the user needs to use two correction plates 4 at the same time, in addition to setting two fixing plates 1 on the ribs, the two fixing plates 1 can also be fixed by using a stacked arrangement of the extension boards 6, or an overlapping arrangement with angles (including angle change of the XY plane and the XZ plane), so as to enable adjustment of orientation in the three-dimensional space to respond to the needs for various inter-rib widths. Of course, it is possible that the positions of the two fixing plates 1 are swapped, or the extension boards 6 of the two fixing plates 1 can be designed to be integrally formed, all of which belong to the scope of the present invention.

For overlapping arrangement of the extension boards 6, fixing can be made with the engagement notch portions 61 or the connection apertures 62 according to the clinical conditions. In the instant embodiment, the engagement notch portions 61 are formed in two sides of the extension board 6. When the extension boards 6 are arranged to overlap each other, it only needs to use a binding structure, such as steel wire or human body absorbable thread, to wrap the two extension boards 6 by simply making use of the engagement notch portions 61 to position the bonding structure, making the binding operation easy and further enhancing the fixing effect (as shown in FIG. 9). In the instant embodiment, the connection aperture 62 is made in the form of multiple perforations arranged side by side in one row on the extension board 6. When the extension boards 6 are arranged in an overlapping form, according to the spacing distance that is actually required by the correction plate 4, the two extension boards 6 are adjusted to have the connection apertures 62 aligned to be penetrated and fixed by the binding structure in a similar way for free adjustment of the spacing distance between the fixing plates 1 (as shown in FIG. 10). In the drawings, the extension board 6 made in an elongated form is taken as an example for illustration, for further expanding the range of spacing distance of the correction plate 4. This also shows that the appearance of the present invention is not limited. In addition, due to the design of the extension board 6, the operation range of the binding structure is concentrated on the extension board 6 and does not involve winding of the ribs, so that even if the binding structure breaks in the future, it is basically fixed to the extension board 6 and will not remain in the human body, which is safer to use.

Thus, the key factors that the connecting and fixing structure of funnel chest correction plate according to the present invention may improve the prior art technology are as follows:

Firstly, the arrangement of the free ends 211 and the opening end 212 of the fixing groove 21 can greatly reduce the risk of accumulation of blood and other filth, thereby providing higher hygiene safety.

Secondly, the fixing plate 1 is provided with the arrangement of the clamp limiting portion 3 and the open end 31, so as to greatly simplify the operation of combining the correction plate 4 with the fixing plate 1 to thereby improve the convenience of disassembly and assembly, and due to the space required for combination being greatly reduced, the size of the surgical wound can be reduced.

Thirdly, the correction plate 4 only needs the adjusting hole 41 to combine with the fixing portion 11 of the fixing plate 1, so that the structural integrity of the correction plate 4 can be maintained, and the structural strength of the correction plate 4 can be greatly improved.

Fourthly, the adjusting hole 41 of the correction plate 4 allows the fastening member 5 to move in the length direction of the correction plate 4 so as to provide a space for movement of the correction plate 4 during breathing or growing of the user.

Fifthly, the thinned design of the extension board 6 helps reduce the overall thickness when two fixing plates 1 are set in a stacked arrangement or an overlapping arrangement, reduce the extent of protruding for inclined stacking, and provide freedom of adjustment of orientation in the three-dimensional space.

Sixthly, the arrangement of the engagement notch portion 61 or the connection aperture 62 makes it possible to enhance the fixing effect by simply applying a binding operation and to freely adjust the distance between the correction plates 4.

I claim:

1. A connecting and fixing structure of a funnel chest correction plate, comprising:
at least one fixing plate;
a stop limiting portion, which is formed in the fixing plate;
a fixing groove, which is defined between the fixing plate and the stop limiting portion;
a plurality of free ends, which are respectively defined in two end surfaces of the fixing groove;
an opening end, which is formed on one side of the fixing groove different from the free ends;
a clamp limiting portion, which is formed on the fixing plate and is located at one side opposite to the stop limiting portion;
an open end, which is defined between the clamp limiting portion and the stop limiting portion;
a fixing portion, which is formed on the fixing plate and is located between the stop limiting portion and the clamp limiting portion;
a correction plate, which is movable through the open end into the fixing groove to be disposed on the fixing plate and clamped and limited by the stop limiting portion and the clamp limiting portion;
an adjusting hole, which is formed in the correction plate and corresponds in position to the fixing portion when the correction plate is disposed on the fixing plate; and
a fastening member, which corresponds to and is combinable with the adjusting hole and the fixing portion.

2. The connecting and fixing structure of the funnel chest correction plate according to claim 1, wherein an extension board extends from one side of the clamp limiting portion and has a thickness less than a thickness from the fixing plate to the stop limiting portion.

3. The connecting and fixing structure of the funnel chest correction plate according to claim 2, wherein the extension board comprises at least one engagement notch portion formed on at least one side thereof.

4. The connecting and fixing structure of the funnel chest correction plate according to claim 2, wherein the extension board is formed with at least one connection aperture.

5. The connecting and fixing structure of the funnel chest correction plate according to claim 1, wherein the adjusting hole is of an elongated form that allows the fastening member to move along a length direction of the correction plate.

6. The connecting and fixing structure of the funnel chest correction plate according to claim 1, wherein the fixing plate is formed with a through-hole portion for positioning on ribs.

7. The connecting and fixing structure of the funnel chest correction plate according to claim 1, wherein the fixing plate comprises a smooth surface located on one side of the stop limiting portion opposite to the fixing groove.
